(19) United States
(12) Patent Application Publication
(10) Pub. No.: US 2005/0085492 A1
(43) Pub. Date: Apr. 21, 2005

(54) Stereoselective process for the synthesis of chiral garft compounds and intermediates

(75) Inventor: Leera Rahman, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, MINC.

(21) Appl. No.: 10/969,680

(22) Filed: Oct. 20, 2004

Related U.S. Application Data
(60) Provisional application No. 60/512,801, filed on Oct. 20, 2003.
(60) Provisional application No. 60/529,144, filed on Dec. 12, 2003.

(51) Int. Cl. A61K 31/519; C07D 487/02

(52) U.S. Cl. 514/264.11; 544/279

(57) ABSTRACT

The present invention describes a stereoselective preparation of derivatives and precursors of molecules having formula 1: 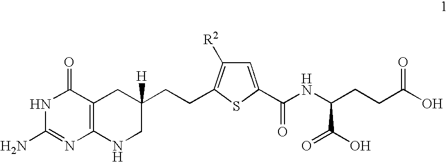 Method of preparing the compounds, intermediates and derivatives are described. The methods described herein allow the preparation of diastereomeric forms of compound having formula 1. The compounds of the invention are GARFT inhibitors.

This application claims the benefit of U.S. Provisional Application No. 60/512,801 filed Oct. 20, 2003, and U.S. Provisional Application No. 60/529,144 filed Dec. 12, 2003, the contents of which are hereby incorporated by reference in their entireties.

### FIELD OF THE INVENTION

The present invention relates to the novel enantioselective synthesis of a GARFT inhibitor containing a methyl substituted thiophene core and intermediates thereof.

### BACKGROUND OF THE INVENTION

The following description of the background of the invention is provided to aid in understanding the invention, but is not admitted to be, or to describe, prior art to the invention. All publications are incorporated by reference in their entirety.

Glycinamide ribonucleotide formyl transferase (GARFT) is a folate dependent enzyme in the de novo purine biosynthesis pathway, a pathway critical to cell division and proliferation. Shutting down this pathway is believed to have an antiproliferative effect, in particular, an antitumor effect. Thus, a number of folate analogs have been synthesized and studied for their ability to inhibit GARFT. A prototypical specific tight binding inhibitor of GARFT, 5,10-dideazatetrahydrofolic acid, has been reported to show antitumor activity. See F. M. Muggia, “Folate antimetabolites inhibitor to de novo purine synthesis,” New Drugs, Concepts and Results in Cancer Chemotherapy, Kluwer Academic Publishers, Boston (1992), 65-87. Compounds that inhibit GARFT consequently inhibit the growth and proliferation of the cells of higher organisms or microorganisms such as bacteria, yeast and fungi.

A large class of antiproliferative agents includes antimetabolite compounds. A particular subclass of antimetabolites known as antifolates or antifoles are antagonists of the vitamin folic acid. Typically, antifolates closely resemble the structure of folic acid and incorporate the characteristic P-benzoyl glutamate moiety of folic acid.

U.S. Pat. No. 5,646,141 (the '141 patent), which is incorporated by reference herein, discloses antiproliferative compounds that inhibit GARFT. The compounds described therein resemble folic acid and incorporate the characteristic glutamate moiety. A process of preparing optically active versions of such GARFT inhibitors is described in U.S. Pat. No. 5,981,748, which is incorporated in its entirety. In view of the costs associated with characterizing racemic compounds, it would be desirable to develop a method for conveniently preparing stereochemically pure forms such that one individual stereoisomer can be prepared and characterized in an efficient and cost-effective manner.

Traditional methods of organic synthesis were often optimized for the production of racemic materials or mixtures of stereoisomers. The production of stereochemically pure material has historically been achieved in one of two ways: use of enantiomerically pure starting materials derived from natural sources (the so-called “chiral pool”); and the resolution of racemic mixtures by classical techniques. Each of these methods has serious drawbacks.

Use of the chiral pool is essentially limited to what is naturally available, and it is often limited to one enantiomer or diastereomer, as in the case of naturally occurring amino acids. Resolution of racemates, which requires the use of resolving agents, may be inconvenient and time-consuming. Furthermore, resolution often means that the undesired enantiomer is discarded, thus decreasing efficiency and wasting half of the material. The present invention overcomes the above disadvantages by providing a process which enables one to obtain stereochemically enriched product directly, without the need for laborious procedures and separations.

### SUMMARY OF THE INVENTION

The present invention concerns methods and compounds useful for the stereoselective synthesis of pharmaceutical agents. In one aspect, the methods and compounds described herein may be used to synthesize L-glutamic acid, N-[[5-[2-[(6S)-2-amino-1,4,5,6,7,8-hexahydro-4-oxopyrido[2,3-d]pyrimin-6-yl]ethyl]-4-methyl-2-thienyl]carbonyl], (1), whose chemical structure is depicted below:

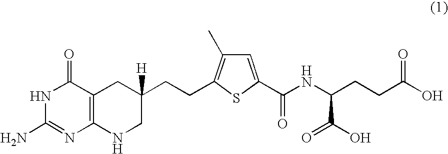

In one aspect are methods for producing a compound or salt of formula (I)-S: 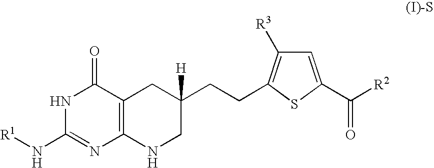
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups; each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
said method comprising:
   (i) treating a compound or salt of formula (II)-S: 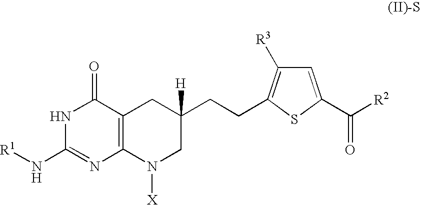
      with an acid in an inert solvent, wherein X is —CHR⁵R⁶ and comprises a chiral entity; R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃-C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In one embodiment of this method X is 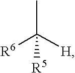
wherein R⁵ is a (C₁-C₃)alkyl, optionally substituted with 1-3 independently selected Y² groups; and R⁶ is an aryl, optionally substituted with 1-3 independently selected Y² groups. In a further embodiment of this method, X is

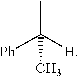

In another embodiment of this method, R¹ is —C(O)R⁷ and R⁷ is selected from the group consisting of (C₁-C₆)alkyl, alkaryl and aryl. In yet a further embodiment of this method, R¹ is

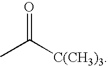

In another embodiment of this method, R² is OR⁴, wherein R⁴ is (C₁-C₆)alkyl, optionally substituted with 1 to 3 independently selected Y¹ groups. In a further embodiment of this method, R⁴ is —CH₂CH₃. In another embodiment of this method, R² is

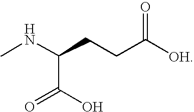

In another aspect is a method of producing a compound or salt of formula (I)-R: 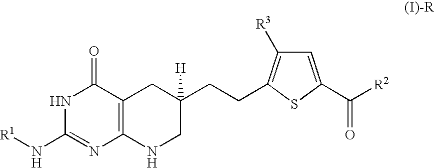
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
said method comprising:
   (i) treating a compound or salt of formula (II)-R: 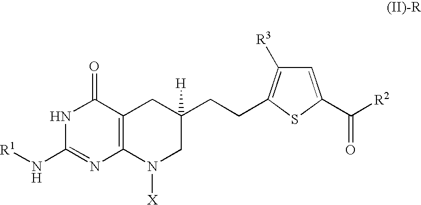
      with an acid in an inert solvent,
wherein X is —CHR⁵R⁶ and comprises a chiral entity;
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In one embodiment of this method, X is 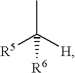
wherein R⁵ is a (C₁-C₃)alkyl, optionally substituted with 1-3 independently selected Y² groups; and R⁶ is an aryl, optionally substituted with 1-3 independently selected Y² groups. In a further embodiment of this method, X is

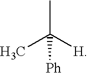

In another embodiment of this method, R¹ is —C(O)R⁷ and R⁷ is selected from the group consisting of (C₁-C₆)alkyl, alkaryl and aryl. In a further embodiment of this method, R¹ is

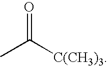

In another embodiment of this method, R² is OR⁴, wherein R⁴ is (C₁-C₆)alkyl, optionally substituted with 1 to 3 independently selected Y¹ groups. In a further embodiment of this method, R⁴ is —CH₂CH₃. In another embodiment of this method, R² is

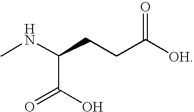

In another aspect of this invention is a compound or salt having formula (II)-S: 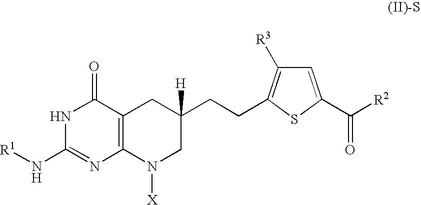
wherein
R¹ is H or an amino protecting group;
R² is —OR⁴ or an amino acid moiety;
R³ and R⁴ are each, independently, H or a moiety selected from the group consisting of (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, Z¹-aryl, and -Z¹-heterocycle, wherein Z¹ is a bond, —C(O)—, —OC(O)—, or —NHC(O)—;
X is —CHR⁵R⁶ and comprises a chiral entity; and
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In yet a further embodiment of such compounds, X is 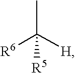
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such compounds, X is

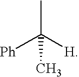

In yet another embodiment of such compounds, R¹ is —C(O)R⁷ and R⁷ is selected from the group consisting of (C₁-C₆)alkyl, alkaryl and aryl. In still a further embodiment of such compounds, R¹ is

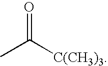

In yet another embodiment of such compounds, R² is OR⁴, wherein R is (C₁-C₆)alkyl, optionally substituted with 1 to 3 independently selected Y¹ groups. In still a further embodiment of such compounds, wherein R⁴ is —CH₂CH₃. In yet another embodiment of such compounds, R² is

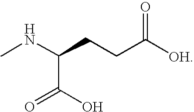

In another aspect of the invention is a compound or salt having formula (II)-R: 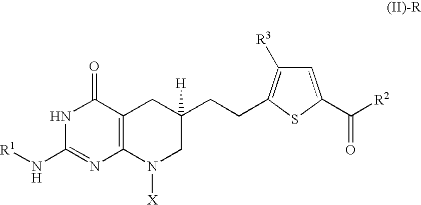
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z₁-aryl, and -Z¹-heterocycle, wherein Z¹ is a bond, —C(O)—, —OC(O)—, or —NHC(O)—;
X is —CHR⁵R⁶ and comprises a chiral entity; and
R⁵ and R⁶ are each, independently selected from the group consisting (C₁-C₆)alkyl, aryl, (CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In yet another embodiment of such compounds, X is 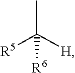
wherein R⁵ is a (C₁-C₃)alkyl, optionally substituted with 1-3 independently selected Y² groups; and R⁶ is an aryl, optionally substituted with 1-3 independently selected Y² groups. IN yet a further embodiment of such compounds, X is

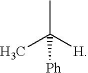

In yet another embodiment of such compounds, R¹ is —C(O)R⁷ and R⁷ is selected from the group consisting of (C₁-C₆)alkyl, alkaryl and aryl. In still a further embodiment of such compounds, R¹ is

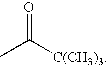

In yet another embodiment of such compounds, R² is OR⁴, wherein R⁴ is (C₁-C₆)alkyl, optionally substituted with 1 to 3 independently selected Y¹ groups. In still a further embodiment of such compounds, R⁴ is —CH₂CH₃. In another embodiment of such compounds, R² is

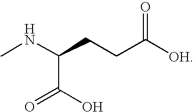

In a further embodiment of this invention are methods of making the compound or salt having formula (II)-S: 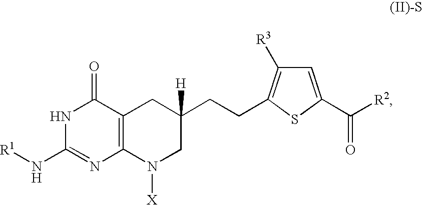
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z₁-(C₁-C₆)dialkylamino, -Z¹-(C¹-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
X is —CHR⁵R⁶ and comprises a chiral entity; and
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group;
said method comprising
(i) treating a compound or salt of formula (III): 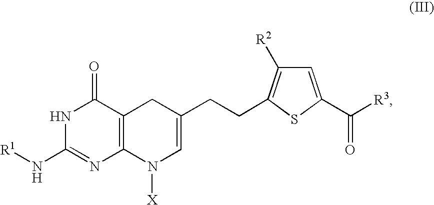
   with dihydrogen in the presence of a catalyst.

In a further embodiment of such methods, the catalyst comprises palladium.

In a further embodiment of such methods, X is 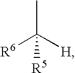
wherein R⁵ is a (C₁-C₃)alkyl, optionally substituted with 1-3 independently selected Y² groups ; and R⁶ is an aryl, optionally substituted with 1-3 independently selected Y² groups. In a still further embodiment of such methods, X is

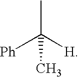

In a further embodiment of such methods, R¹ is —C(O)R⁷ and R⁷ is selected from the group consisting of (C₁-C₆)alkyl, alkaryl and aryl. In still a further embodiment of such methods, R¹ is

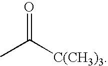

In a further embodiment of such methods, R² is OR⁴, wherein R⁴ is (C₁-C₆)alkyl, optionally substituted with 1 to 3 independently selected Y¹ groups. In still a further embodiment of such methods, R⁴ is —CH₂CH₃.

In another aspect of the invention are methods of making the compound or salt having formula (II)-R: 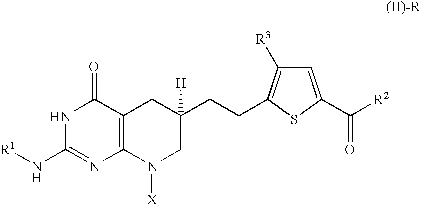
comprising:
(i) treating a compound or salt of formula III: 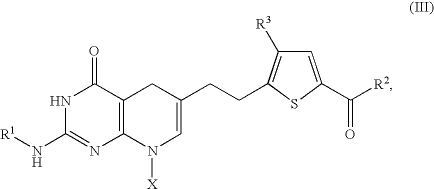
   with dihydrogen in the presence of a catalyst.

In a further embodiment of such methods, the catalyst comprises palladium.

In a further embodiment of such methods, X is 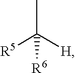
wherein R⁵ is a (C₁-C₃)alkyl, optionally substituted with 1-3 independently selected Y² groups; and R⁶ is an aryl, optionally substituted with 1-3 independently selected Y² groups.

In still a further embodiment of such methods, X is

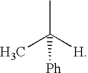

In a further embodiment of such methods, R¹ is —C(O)R⁷ and R⁷ is selected from the group consisting of (C₁-C₆)alkyl, alkaryl and aryl. In still a further embodiment of such methods, R¹ is

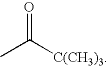

In yet a further embodiment of such methods, R² is OR⁴, wherein R⁴ is (C₁-C₆)alkyl, optionally substituted with 1 to 3 independently selected Y¹ groups. In still a further embodiment of such methods, R⁴ is —CH₂CH₃.

In a further aspect of this invention are compounds or salts having formula (III): 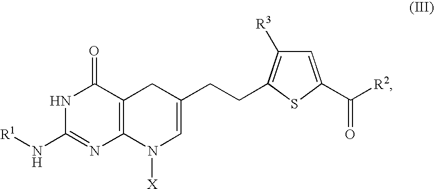
wherein
R¹ is H or an amino protecting group;
R² is R⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
X is —CHR⁵R⁶ and comprises a chiral entity;
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In a further embodiment of such compounds, X is 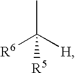
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such compounds, X is

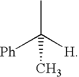

In another embodiment of such compounds, X is 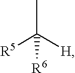
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such compounds, X is

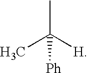

In another embodiment of such compounds, R¹ is —C(O)R⁷ and R⁷ is selected from the group consisting of (C₁-C₆)alkyl, alkaryl and aryl. In still a further embodiment of such compounds, R¹ is

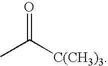

In another embodiment of such compounds, R² is OR⁴, wherein R⁴ is (C₁-C₆)alkyl, optionally substituted with 1 to 3 independently selected Y¹ groups. In yet a further embodiment of such compounds, R⁴ is —CH₂CH₃.

In another aspect of this invention are methods of producing a compound or salt having formula III: 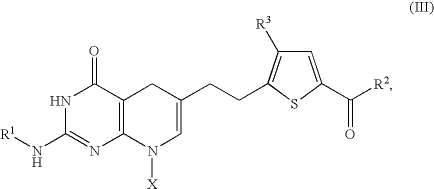
comprising:
(i) treating a compound or salt of formula (IV): 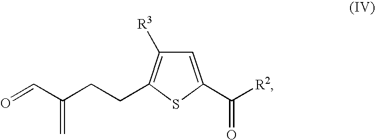
   with a compound or salt of formula (V): 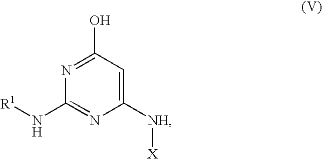
   and with an acid in a solvent.

In another embodiment of such methods, X is 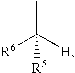
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such methods, X is

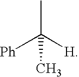

In another embodiment of such methods, X is 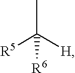
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such methods, X is

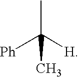

In another embodiment of such methods, R¹ is —C(O)R⁷ and R⁷ is selected from the group consisting of (C₁-C₆)alkyl, alkaryl and aryl. In a still further embodiment of such methods, R¹ is

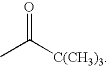

In another embodiment of such methods, R² is OR⁴, wherein R⁴ is (C₁-C₆)alkyl, optionally substituted with 1 to 3 independently selected Y¹ groups.

In another embodiment of such methods, the acid is an acid salt of a secondary amine. In still a further embodiment of such methods, the acid is present in a catalytic amount. In still a further embodiment of such methods is the trifluoroacetic acid salt of N-methyl aniline.

In a further aspect of this invention are compounds or salts having formula (IV): 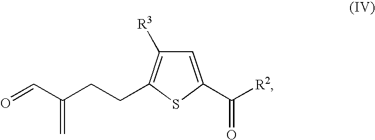
wherein
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups; each Y¹ is independently selected from the group consisting of halogen, cyano, nitro, azido, —OH, —NH₂, -Z₁-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy. -Z¹-(C₃₋C₆)cycloalkyl, -Z¹-aryl, and -Z¹-heterocycle, wherein Z¹ is a bond, —C(O)—, —OC(O)—, or —NHC(O)—.

In a further embodiment of such compounds, R² is OR⁴, wherein R⁴ is (C₁-C₆)alkyl, optionally substituted with 1 to 3 independently selected Y¹ groups. In still a further embodiment of such compounds, R⁴ is —CH₂CH₃.

In a further aspect of this invention are methods of producing a compound or salt having formula (IV): 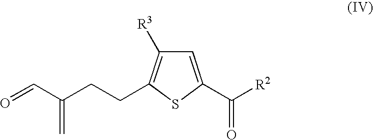
comprising:
(i) treating a compound or salt of formula (VI): 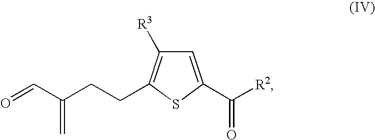
   with a tertiary base and N,N-dimethylmethyleneammonium iodide.

In a further embodiment of such methods, the tertiary base is triethylamine.

In a further embodiment of such methods, R² is OR⁴, wherein R⁴ is (C₁-C₆)alkyl, optionally substituted with 1 to 3 independently selected Y¹ groups. In still a further embodiment of such methods, R⁴ is —CH₂CH₃.

In a further aspect of this invention are compounds having formula (V): 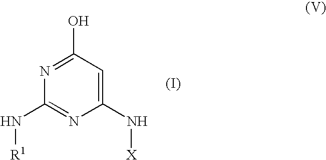
wherein
R¹ is H or an amino protecting group;
X is —CHR⁵R⁶ and comprises a chiral entity; and
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

In a further embodiment of such compounds, X is 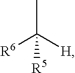
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such compounds, X is

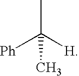

In another embodiment of such compounds, X is 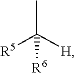
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such compounds, X is

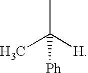

In a further embodiment of such compounds, R¹ is —C(O)R⁷ and R⁷ is selected from the group consisting of (C₁-C₆)alkyl, alkaryl and aryl. In still a further embodiment of such compounds, R¹ is

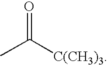

In a further aspect of this invention are methods of producing compounds or salts having formula (V): 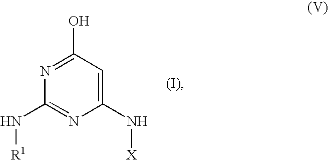
wherein R¹ is H comprising
(i) treating a compound or salt of formula (VII): 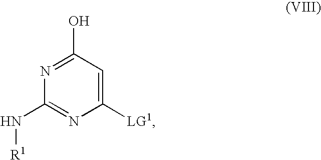
   wherein LG¹ is a leaving group,
with an amine, NH₂—X.

In a further embodiment of such methods, LG¹ is selected from the group consisting of Cl, Br, and I.

In a further embodiment of such methods, X is 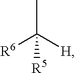
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such methods, X is

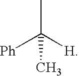

In a further embodiment of such methods, X is 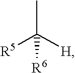
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such methods, X is

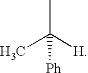

In a further aspect of this invention are compounds having formula (VI): 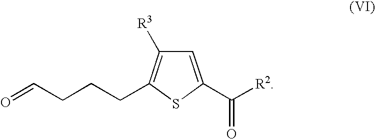
wherein
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, —Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—.

In a further embodiment of such compounds, R² is OR⁴, wherein R⁴ is (C₁-C₆)alkyl, optionally substituted with 1 to 3 independently selected Y¹ groups. In still a further embodiment of such compounds, R⁴ is —CH₂CH₃.

In a further aspect of this invention are methods of producing a compound or salt having formula (VI): 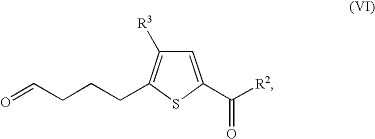
comprising
(i) treating a compound or salt of formula (VII): 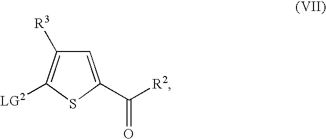
   wherein LG² is a leaving group,
with 3-butenol, a base, and a catalyst.

In a further embodiment of such methods, LG² is selected from the group consisting of Cl, Br and I.

In a further embodiment of such methods, R² is OR⁴, wherein R⁴ is (C₁-C₆)alkyl, optionally substituted with 1 to 3 independently selected Y¹ groups. In still a further embodiment of such methods, R⁴ is —CH₂CH₃.

In a further aspect of this invention are methods of producing the compound or salt having formula (V): 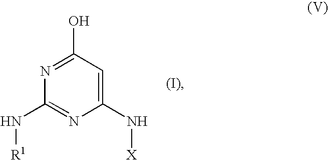
wherein R¹ is an amino protecting group;
comprising
   (i) treating an amount of compound or salt of formula (IX): 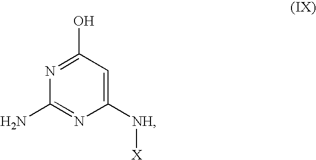
      with a solvent, a tertiary amine, and R⁷C(O)OC(O)R⁷, and R⁷ is selected from the group consisting of (C₁-C₆)alkyl, alkaryl and aryl.

In a further embodiment of such methods, X is 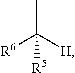
wherein R⁵ is a (C₁-C₃)alkyl, optionally substituted with 1-3 independently selected Y² groups; and R⁶ is an aryl, optionally substituted with 1-3 independently selected Y² groups. In yet a further embodiment of such methods, X is

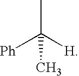

In a further embodiment of such methods, X is 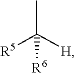
wherein R⁵ is a (C₁-C₃)alkyl, optionally substituted with 1-3 independently selected Y² groups; and R⁶ is an aryl, optionally substituted with 1-3 independently selected Y² groups. In still a further embodiment of such methods, X is

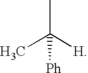

In a further embodiment of such methods, R⁷ is —C(CH₃)₃. In another aspect of the invention are salts having the formula IIIa: 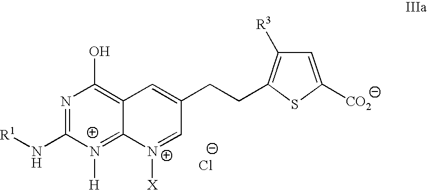
wherein R¹ is H or an amino protecting group;
R³ is H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
X is —CHR⁵R⁶ and comprises a chiral entity; and
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂; -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

The term “acyl group” is to be understood in the broadest sense in connection with the present process. It includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl and butyryl; aralkanoyl, such as phenylacetyl; aroyl, such as benzoyl and toluyl; aryloxyalkanoyl, such as POA; alkoxycarbonyl, such as methoxycarbonyl, ethoxycarbonyl, 2,2,2-trichloroethoxycarbonyl, BOC (tert-butoxycarbonyl) and 2-iodoethoxycarbonyl; aralkoxycarbonyl, such as CBZ (“carbobenzoxy”), 4-methoxybenzyloxycarbonyl and FMOC; and arylsulfonyl. Preferred amino-protecting groups are BOC and, furthermore CBZ, Fmoc, benzyl and acetyl.

The term “alkyl”, as used herein, unless otherwise indicated, includes saturated monovalent hydrocarbon radicals having straight or branched moieties.

The term “ambient temperature” means temperature condition typically encountered in a laboratory setting. The includes the approximate temperature range about 20-30° C.

The term “amino protecting group” as used herein refers to groups which are suitable for protecting (blocking) an amino group against chemical reactions, but which are easy to remove after the desired chemical reaction has been carried out elsewhere in the molecule. Typical of such groups are, in particular, unsubstituted or substituted acyl, aryl, aralkoxymethyl or aralkyl groups. A preferred amino protecting group is —C(O)R, wherein R is (C₁-C₆)alkyl, alkaryl and aryl. Some specific examples of amino protecting groups include trichloroethoxycarbonyl, benzyloxycarbonyl (Cbz), chloroacetyl, trifluoroacetyl, phenylacetyl, formyl, acetyl, benzoyl, tert-butoxycarbonyl (Boc), para-methoxybenzyloxycarbonyl, diphenylmethoxycarbonyl, phthaloyl, succinyl, benzyl, diphenylmethyl, triphenylmethyl (trityl), methanesulfonyl, para-toluenesulfonyl, pivaloyl, trimethylsilyl, triethylsilyl, triphenylsilyl, and the like.

The term “aqueous base” refers to any organic or inorganic base. Preferred aqueous bases include metal bicarbonates, for example sodium bicarbonate, potassium carbonate, cesium carbonate, triethylamine (TEA), diisopropylethylamine (DIPEA) and the like.

The term “aromatic solvent” means benzene, toluene, xylene isomers or mixtures thereof, and the like.

The term “aryl”, as used herein, unless otherwise indicated, includes an organic radical derived from an aromatic hydrocarbon by removal of one hydrogen, such as phenyl or naphthyl.

The term “asymmetric induction” as used herein means the preferential formation in a chemical reaction of one enantiomer or diastereomer over another as a result of the influence of a chiral feature present in the substrate, reagent, catalyst, or environment.

The term “chiral entity” refers to any chemical moiety having one or more chiral centers, plane of chirality, or any recognized chiral feature. A chiral molecule is one that is not superimposable on its mirror image.

The term “detectable amount” as used herein is an amount or amount per unit volume that can be detected using conventional techniques, such as ¹H and ¹³C NMR, HPLC, FT-IR, Raman spectroscopy, mass spectroscopy and the like.

The term “diastereomer” as used herein refers to any pair of stereoisomers not related as an object and its mirror image. “Diastereoisomers” are stereoisomers that have at least two chiral centers, but which are not mirror-images of each other. Compounds that have two chiral centers exist as two diastereomers. The diastereomers can be separated by conventional methods such as crystallization, distillation or chromatography. Each separate diastereomer exists as a pair of enantiomers which can be separated by conventional methods such as those described above. Alternatively, the diastereomers or enantiomers can be prepared separately, by means of stereospecific or diastereoselective reactions known to those skilled in the art.

The term “enantiomer” as used herein refers to a pair of stereoisomers related as an object and its mirror image. “Enantiomers” are a pair of stereoisomers that are non-superimposable mirror images of each other. A 1:1 mixture of a pair of enantiomers is a “racemic” mixture. The term “(.+−.)” is used to designate a racemic mixture where appropriate.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo 1.0}hexanyl, 3-azabicyclo 1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following: 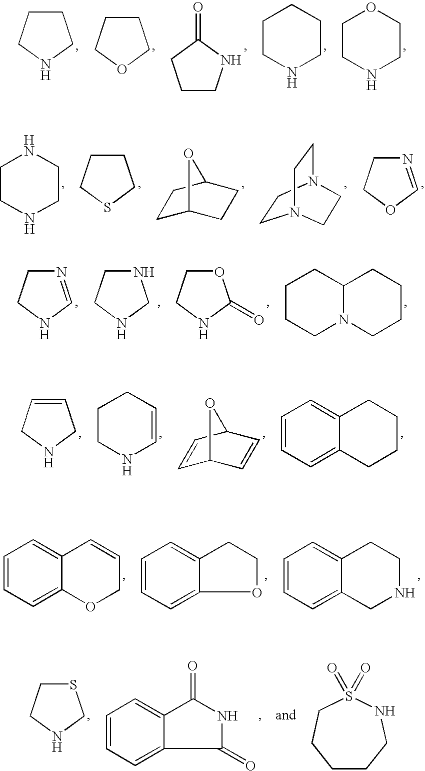
Unless otherwise indicated, the term “oxo” refers to ═O.

The term “halo”, as used herein, unless otherwise indicated, means fluoro, chloro, bromo or iodo. Preferred halo groups are fluoro, chloro and bromo.

The term “isolating” as used herein refers to the purification of compounds and intermediates using any method or technique familiar to a skilled chemist. “Isolation” can be effected, if desired, by any suitable separation or purification procedure such as, for example, filtration, extraction, crystallization, (chiral) column chromatography, thin-layer chromatography or preparatory chromatography, or a combination of these procedures. Specific illustrations of suitable separation and isolation procedures can be had by reference to the Examples. However, other equivalent separation or isolation procedures can, of course, also be used. Except as specified to the contrary, the compounds and intermediates are isolated and purified by conventional means.

The term “isomer” refers to different compounds that have the same molecular formula.

The term “leaving group” as used herein refers to any moiety or atom that is susceptible to nucleophilic substitution or elimination. Typically, these are atoms or moieties that when removed by nucleophilic substitution or elimination are stable in anionic form. Examples of leaving groups useful in the present invention include alkyl or arylsulphonate groups such as tosylate, brosylate, mesylate or nosylate, or halides such as chloride, bromide, or iodide.

The term “minimal amount” means the least amount of solvent required to completely dissolve a substance at a given temperature.

The term “optically pure” is intended to mean a compound which consists of at least a sufficient amount of a single enantiomer (or diastereomer in the case of plural chiral centers) to yield a compound having the desired pharmacological activity. Preferably, “optically pure” is intended to mean a compound that consists of at least 90% of a single isomer (80% enantiomeric or diastereomeric excess), preferably at least 99% (98% e.e. or d.e.), more preferably at least 95% (90% e.e. or d.e.), even more preferably at least 97.5% (95% e.e. or d.e.), and most preferably at least 99% (98% e.e. or d.e.).

The term “pharmaceutically acceptable, carrier, diluent, or vehicle” as used herein may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

The term “pharmaceutically acceptable salt” or “salt” refers to a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salts. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, para-toluene sulfonates (tosylates), formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, y-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The term “racemic” refers to chemical mixtures having equal amounts of enantiomeric molecules present together. The material is termed racemic independently of whether it is crystalline, liquid, or gaseous.

The term “recrystallize” as used herein refers to the process of completely dissolving a solid in a first solvent with heating if necessary, and then inducing precipitation, usually by cooling the solution, or by adding a second solvent in which the solid is poorly soluble.

The term “regioselectivity” as used herein refers to reactions in which bonds can be made or broken in two or more different orientations. If one orientation is significantly favored, the reaction is regioselective.

The term “separating from” as used for example in a synthesis description, comprise any of the following steps, filtering, washing with extra solvent or water, drying with heat and or under vacuum.

The term “acid” refers to both protic and nonprotic acids (such as hydrogen halide solution (e.g. HCL), methanesulfonic acid, sulfuric acid, trifluoroacetic acid or phosphoric acid). Preferably anhydrous acids are used in the invention, where the anhydrous acid is an acid salt of a secondary amine, i.e. trifluoroacetic acid salt of N-methyl aniline.

The following terms as used in this invention are as defined as follows: “Me” represents methyl or CH₃—, “Et” represents ethyl or CH₃CH₂—, “Bu” represent butyl, “OAc” represents “—O—C(O)—CH₃”, “Pd” represents palladium, “Ts” represents tosyl, “Ph” represents phenyl, “piv” represents pivaloyl, “Glu” represents glutamate, “O^{t}Bu” represents tert-butoxy, “CDI” represents carbonyldiimidazole, and “NMP” represents and N-methyl pyridine. In addition the terms “h” or hr” represents hour(s), “min” or “mins” represents minute(s), and “conc” represents “concentrated”.

The terms “solvent”, “inert organic solvent” or “inert solvent” mean a solvent inert under the conditions of the reaction being described in conjunction therewith [including, for example, benzene, toluene, sulfolane, acetonitrile, tetrahydrofuran (“THF”), tetrahydropyran (THP), trifluoroacetic acid (TFA), tert butyl methyl ether (MTBE), dimethylformamide (“DMF”), chloroform, methylene chloride (or dichloromethane), diethyl ether, methanol, 1-propylalcohol (1-PrOH), 1-butanol (1-BuOH), ethyl acetate (EtOAc), pyridine (pyr), dimethyl-tert-butyl silyl (TBDM), O-(7-Azabenzotriazol-1-yl)-N,N,N′,N′-tetramethyluronium hexafluorophosphate (HATU), mixtures thereof and the like]. Unless specified to the contrary, the solvents used in the reactions of the present invention are inert organic solvents.

Additional terms as used in the invention are defined as follows:

The term “slurry” refers to a solid substance suspended in a liquid medium, typically water or an organic solvent.

The term “stereoisomer” refers to molecules which have the same connectivity of atoms but differ in the way atoms or groups of atoms are oriented in space. The absolute stereochemistry is specified according to the Cahn-Ingold-Prelog R—S system. When the compound is a pure enantiomer the stereochemistry at each chiral carbon may be specified by either R or S. Resolved compounds whose absolute configuration is unknown are designated (+) or (−) depending on the direction (dextro- or levorotary) which they rotate the plane of polarized light at the wavelength of the sodium D line.

The term “stereospecific” as used herein, means a reaction where starting materials differing only in their spacial configuration are converted to stereoisomerically distinct products. For example, in a stereospecific reaction, if the starting material is enantiopure (100% enantiomer excess “ee”), the final product will also be enantiopure. Similarly if the starting material has an enantiomer excess of about 50%, the final product will also have about a 50% enantiomer excess. “Enantiomer excess” as used herein refers to the mole percent excess of a single enantiomer over the racemate.

The term “stereoselective” as used herein refers to chemical reactions in which diastereomerically different materials may be formed or destroyed during the course of a reaction. Stereoselective reactions are those in which one diastereomer (or one enantiomeric pair of diastereomers) is formed or destroyed in considerable preference to others that might be formed or destroyed. Stereoselective reactions may be characterized as being “partially stereoselective, for example 90 percent stereoselective, 60 percent-stereoselective.

The term “substantially free of other forms” refers to an amount of substance having a purity of greater than 90-99.9%

The term “suitable transition metal catalyst” as used herein comprises any of a family of hydrogenation catalysts. Fe, Ru, Os, Co, Rh, Ir, Ni, Pd, Pt. Hydrogenation catalysts may be soluble or insoluble homogeneneous or hetergeneous. Hydrogenation catalysts may have a chiral auxiliary itself as is well know for example chiral rhodium, ruthenium catalysts.

The term “therapeutically effective amount” refers to the amount of an active agent of the invention that may be used to treat diseases mediated by modulation or regulation of protein kinases. An “effective amount” is intended to mean that amount of an agent that significantly inhibits proliferation and/or prevents de-differentiation of a eukaryotic cell, e.g., a mammalian, insect, plant or fungal cell, and is effective for the indicated utility, e.g., specific therapeutic treatment.

Certain compounds of Formula (I) may have asymmetric centers and therefore exist in different enantiomeric forms. All optical isomers and stereoisomers of the compounds of Formula (I), and mixtures thereof, are considered to be within the scope of the invention. With respect to the compounds of Formula (I), the invention includes the use of a racemate, one or more enantiomeric forms, one or more diastereomeric forms, or mixtures thereof. Although the compounds of Formula I are shown in the 4-oxo form and are referred to such by name, the oxo groups exists in tautomeric equilibrium with corresponding 4-hydroxy tautomer as shown below. This invention relates to the use of all such tautomers and mixtures thereof. The following pairs of tautomeric structures (each connected by arrows) are considered to be equivalent unless otherwise noted.

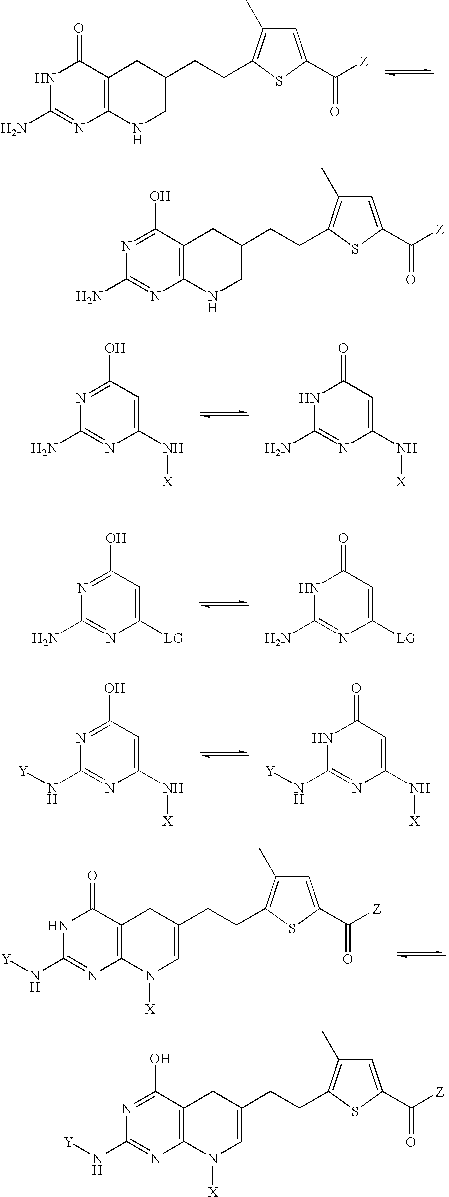

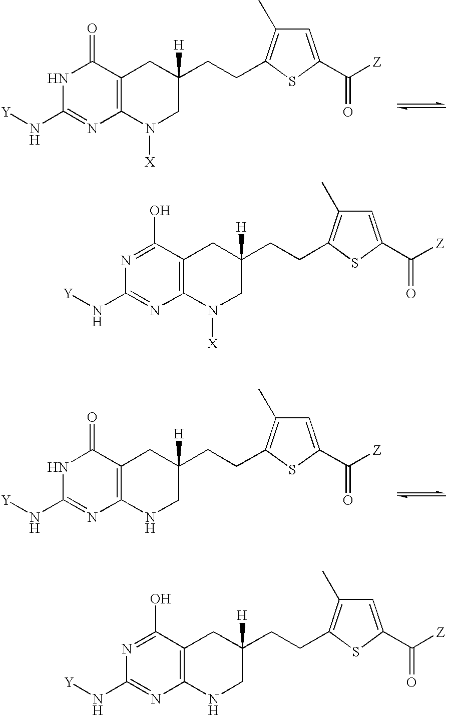

Certain functional groups contained within the compounds of the present invention can be substituted for groups which have similar spatial or electronic requirements as the parent group, but exhibit differing or improved physicochemical or other properties. Suitable examples are well known to those of skill in the art, and include, but are not limited to moieties described in Patini et al., Chem. Rev. 1996, 96, 3147-3176 and references cited therein.

The subject invention also includes isotopically-labelled compounds, which are identical to those recited in Formula (I), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e.,²H can afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples and Preparations below, by substituting a readily available isotopically labelled reagent for a non-isotopically labelled reagent.

Other aspects, advantages, and preferred features of the invention will become apparent from the detailed description below.

### DETAILED DESCRIPTION OF THE INVENTION

The reactions set forth below were generally carried out under a positive pressure of argon or nitrogen or with a drying tube, at ambient temperature (unless otherwise stated), in anhydrous solvents, and the reaction flasks were fitted with rubber septa for the introduction of substrates and reagents via syringe. Analytical thin layer chromatography (TLC) was performed on glass-backed silica gel 60 F 254 plates Analtech (0.25 mm) and eluted with the appropriate solvent ratios (v/v), and are denoted where appropriate. The reactions were assayed by TLC and terminated as judged by the consumption of starting material.

Work-ups were typically done by doubling the reaction volume with the reaction solvent or extraction solvent and then washing with the indicated aqueous solutions using 25% by volume of the extraction volume unless otherwise indicated. Product solutions were dried over anhydrous MgSO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Baker grade flash silica gel (47-61 □m) and a silica gel: crude material ratio of about 20:1 to 50:1 unless otherwise stated. Hydrogenolysis was done at the pressure indicated in the examples or at ambient pressure.

¹H-NMR spectra were recorded on a Bruker instrument operating at 300 MHz and ¹³C-NMR spectra were recorded operating at 75 MHz. NMR spectra were obtained as CDCl₃ solutions (reported in ppm), using chloroform as the reference standard (7.25 ppm and 77.00 ppm) or CD₃OD (3.4 and 4.8 ppm and 49.3 ppm), or internally tetramethylsilane (0.00 ppm) when appropriate. Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### General Synthetic Schemes Used for the Preparation of Thienopyridine Compounds

Preparative Methods

The following schemes depict methods describing typical synthetic procedures using specific materials. Many embodiments of the present invention may be synthesized using the described methods. The skilled artisan will recognize that different acids, acid chlorides, amines, alcohols, bases, solvents and derivatives may be substituted in the following descriptions to suit the preparation of a desired embodiment. The following schemes may be scaled upwards or downwards to suit the amount of desired material.

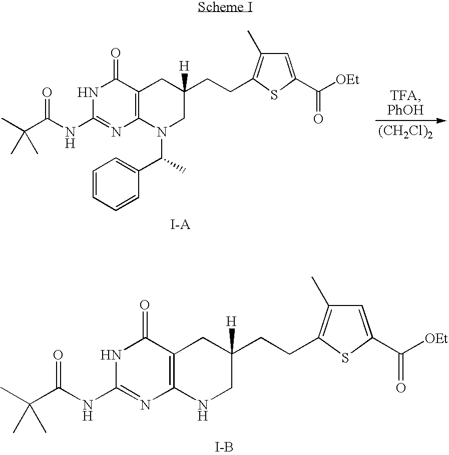

Scheme I depicts the removal of chiral auxiliary in intermediate I-A to yield amino protected I-B. The preparation of intermediate I-A is depicted and described in Scheme II. The further deprotection and functionalization of I-B is depicted and described in Schemes VIII-X.

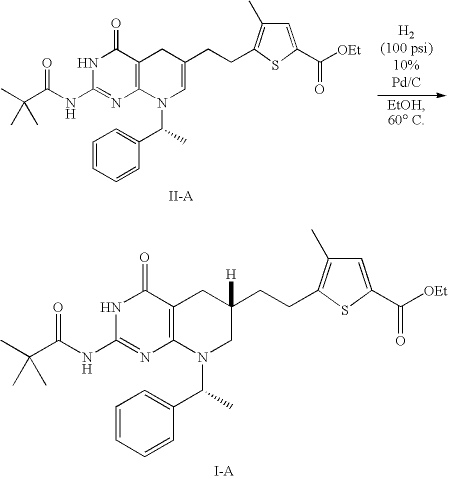

Scheme II depicts a diastereoselective hydrogenation of enamine II-A using Pd/C catalyst to yield predominantly the diastereomer shown as I-A. In Scheme II, H₂ addition occurs predominantly to the face shown. It should also be noted that, alternatively, zwitterions of II-A can be hydrogenated as well, in which case the substrate is not the enamine but the pyridinium salt.

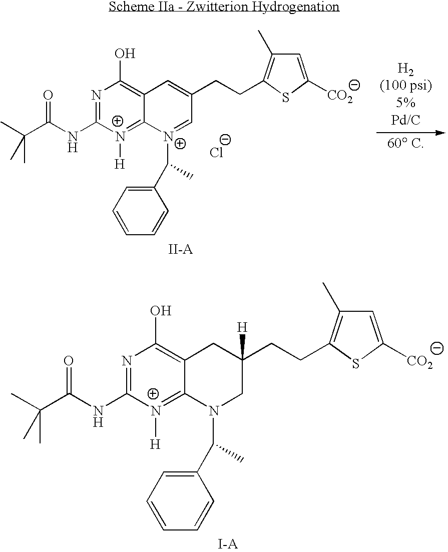

The preparation of intermediate II-A is described in Scheme III. The deprotection of intermediate I-A is depicted in Scheme I.

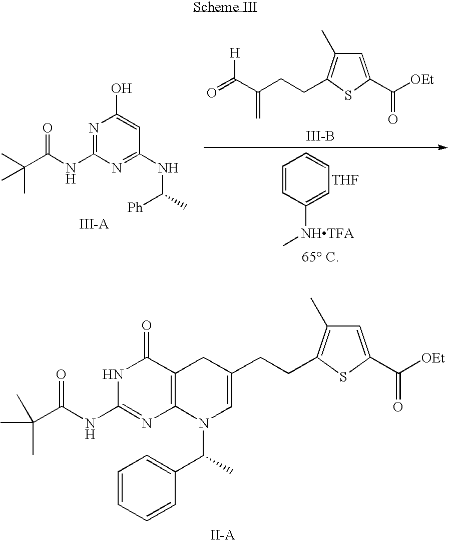

Scheme III depicts treating intermediate aminopyrimidine III-A with intermediate enal III-B with N-methyl aniline TFA salt in THF solvent to yield aminopyrimidinone intermediate II-A. The hydrogenation of Intermediate II-A is depicted and described in Scheme II. The preparation of intermediate III-B is depicted and described in Schemes IV and V. The preparation of intermediate III-A is depicted and described in Schemes VI and VII.

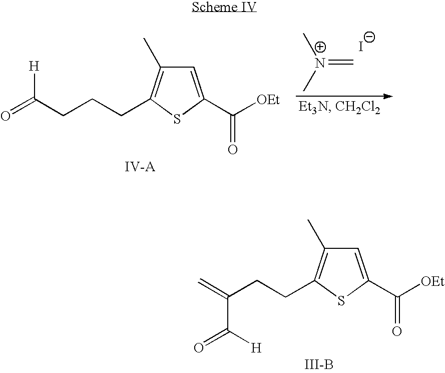

Scheme IV depicts the treatment of aldehyde IV-A with N,N-dimethylmethyleneammonium iodide (Eschenmoser's salt) to yield enal intermediate, III-B. N,N-dimethylmethyleneammonium iodide is commercially available. The further use of intermediate II-B in the present invention is depicted and described in Scheme III. Scheme V depicts and describes the synthesis of intermediate IV-A.

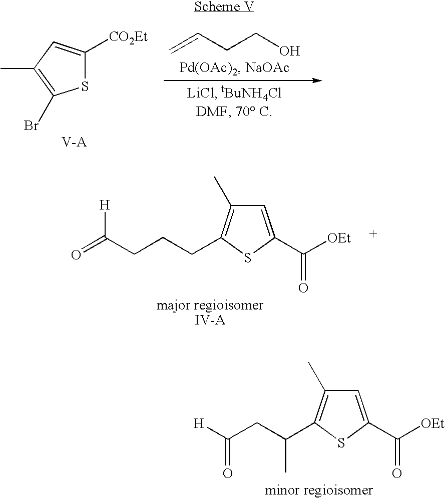

Scheme V depicts a palladium-catalyzed coupling of commercially available bromothiephene V-A (Albemarle) with 3-buten-1-ol (homoallylic alcohol). The initial product of the coupling is a vinyl-thiophene intermediate having a pendant alcohol moiety (not shown). Coupling of an alkene such as buten-1-ol to the thiophene can occur in two regiochemically distinct ways giving rise to major (IV-A) and minor regioisomers. Subsequent double bond isomerization yields the major and minor diastereomers shown. The combined yield of two aldehydes is 85% with 90% regioselectivity for the desired regioisomer, IV-A.

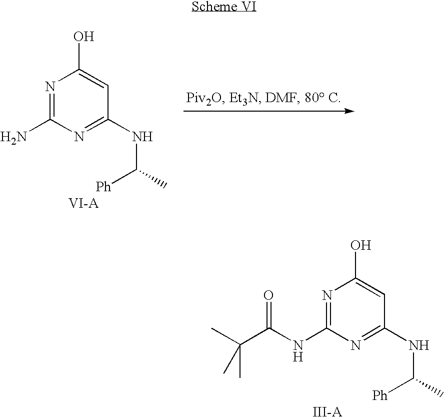

Scheme VI depicts the protection of the pyrimidine amino group of intermediate VI-A using trimethylacetic anhydride, (abbreviated Piv₂O). The primary amino group of VI-A reacts with one electrophilic carbonyl moiety of Piv₂O, leading to formation of chiral N-Piv aminopyrimidinone III-A in 90% yield. One equivalent of acid is co-produced in Scheme VI (not shown) which is effectively neutralized by the base NEt₃. The latter base is effective because it does not compete with amine moiety on the pyrimidine ring.

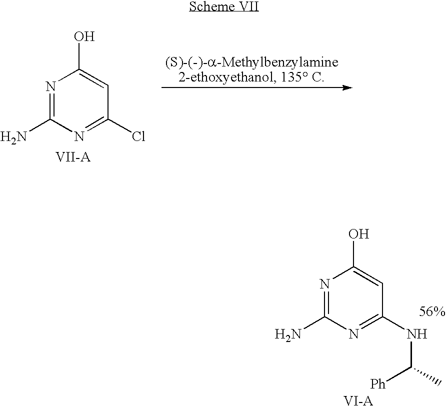

Scheme VII depicts the addition of a chiral amine to commercially available chloro pyrimidine VII-A to form a chiral N-Piv aminopyrimidinone VI-A. The amino moiety of chiral (S)-(−)-α-methylbenzylamine displaces the leaving group chloride from VII-A to make a intermediate VI-A. A number of commercially available chiral amines (or HCL salt precursors) may be used in place of methylbenzylamine. These are described elsewhere herein.

Schemes VIII-X depict the final steps in the complete synthesis of one embodiment of the present invention. These steps comprise the deprotection of I-B and its subsequent functionalization.

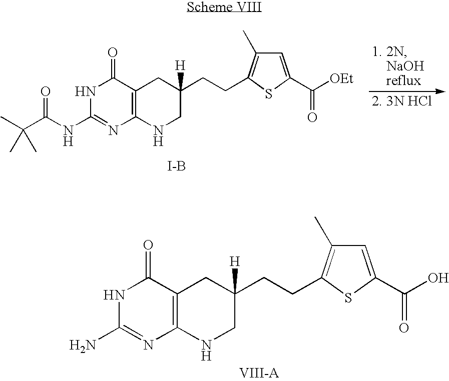

Scheme VIII depicts a deprotection step used to remove the ester and amino protecting groups from intermediate I-B. See Scheme I for the preparation of intermediate I-B. The functionalization of intermediate VIII-A is further depicted and described in Scheme IX.

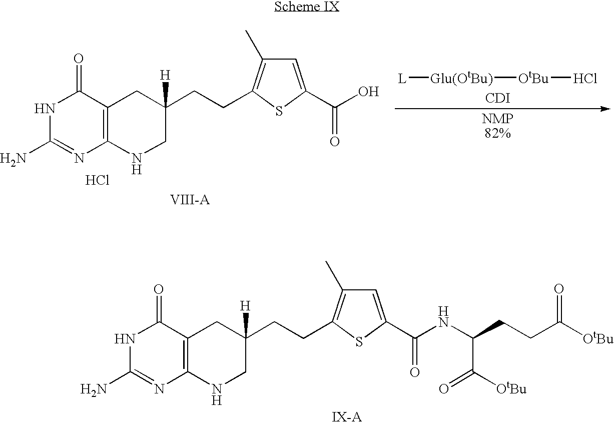

Scheme IX depicts the coupling of a protected amino acid (glutamate) to intermediate VIII-A. This coupling scheme is general and may be used to couple any protected amino acid to a carboxylate moiety.

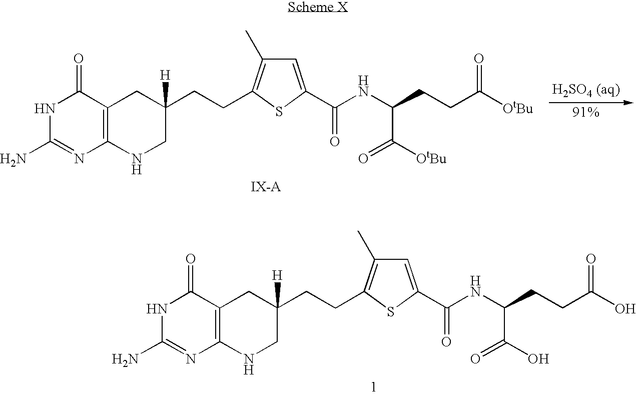

Scheme X depicts the final deprotection step of intermediate IX-A to give the depicted diastereomer of the invention, 1.

Compounds having a structural formula 1: 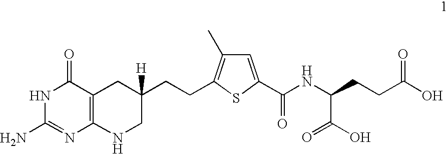
may be prepared in high stereospecificity according to methods disclosed herein. Compound 1 exhibits two carbon chiral centers, one center within the pyridopyrimidone ring indicated by an “H” with a solid wedge pointing “up”, and a second located at the “alpha” carbon of the pendant amino acid side chain (and also indicated with a solid wedge). Compound 1 as drawn has at least three related stereoisomers (more than four if permanent rotomers exist). They are explicitly shown below:

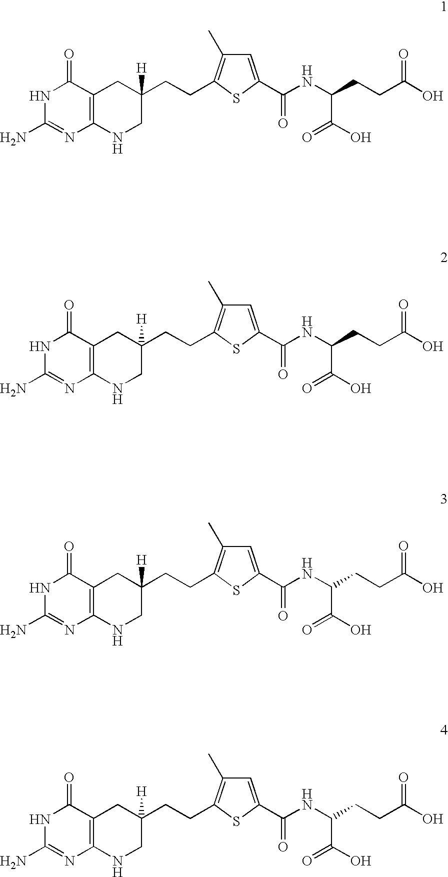

The written description disclosed herein describes the synthesis of compounds 1 and 2. However, the skilled artisan will recognize that the methods disclosed herein can be extended to include compounds 3 and 4, by simply substituting the unnatural or “D” amino acids. Such amino acids and methods for making them are known in the literature.

Compound 1 (or 2, 3, and 4) may be obtained according to the present invention by the acid catalyzed hydrolysis of their corresponding acid esters as shown in Scheme X. The hydrolysis is shown for the t-butyl esters, however the skilled artisan will recognize that any hydrolyzable ester which yields the carboxylic acid, is equivalent. The ester groups are present on the amino acid moiety because they serve to protect the acid moieties in the previous step, exemplified in Scheme IX. In Scheme IX, the free amino group of the amino acid is coupled to the carboxylic acid moiety of compound VIII-A. In the absence of the ester protecting groups, “self coupling” might occur under the conditions employed in Scheme X.

The coupling chemistry of Scheme IX may be used to couple other amino acids Or indeed other amine-bearing compounds to the intermediate VIII-A. Intermediate VII-A is in turn obtained by base-catalyzed hydrolysis of the amino- protected intermediate I-B depicted in Scheme VIII. The particular amine protecting group was found to give reproducible results, however, it is merely representative of a class of amine protecting groups.

Intermediate I-B having the structural formula: 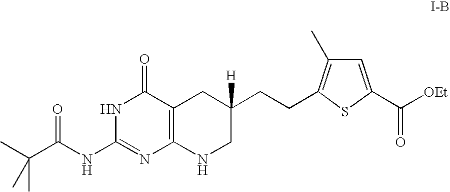
may be synthesized according to Scheme I, by the acid catalyzed cleavage of a chiral amino group present in the compound or salt of formula I-A:

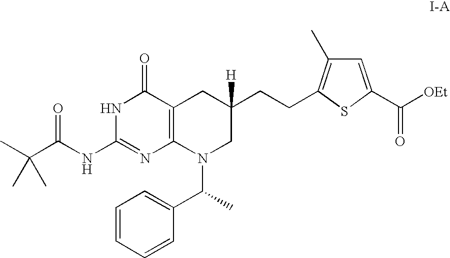

The particular conditions employed in the Example described herein (See Example I) are merely representative and the skilled artisan will recognize at once equivalent solvents and acids.

Intermediate I-A may be obtained by the hydrogenation of enamine intermediate II-A according to Scheme II. Intermediate I-A having formula: 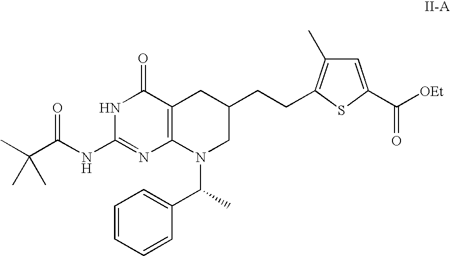
has only one double bond susceptible to hydrogenation. Addition of dihydrogen to this double bond can occur to either “face” of the alkene.

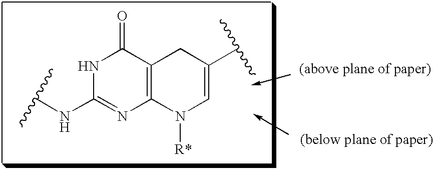

In the presence of a chiral directing group, for example the methylbenzyl group of II-A, the two faces become inequivalent and addition to one face or the other may be favored on steric or stereoelectronic grounds. This is an example of asymmetric induction, wherein one chiral center influences the formation of another. In one aspect of the present invention, using the chiral entity at nitrogen shown in Scheme II leads predominantly to the stereoisomer I-A shown in Scheme II. The isomer shown is favored in 6:1 ratio of diastereomers 1 and 2. When the other methylbenzyl enantiomer is used, the diastereomeric ratio is reversed, giving predominantly 2. The skilled artisan will recognize that changing the nature of the chiral entity will influence the observed stereoselectivity of the hydrogenated product. In addition, the parameters used in the hydrogenation (temperature, H₂, pressure, catalyst, catalyst loading, solvent) have given reliable results for the chiral entity shown but are merely exemplary, and could change in other embodiments of the invention.

The preparation of intermediate II-A is described in Scheme III. Intermediate II-A is obtained via an alkylation/cyclization reaction depicted in Scheme III. The overall reaction in Scheme III may occur in stepwise fashion, however, reliable results have been obtained using the procedure described in Example III. Nonetheless, the order of addition of reagents may affect the yield as well as the relative amount of the added acid N-methylamine TFA salt. Indeed, omission of the N-methyl amine TFA salt has been found to lead to some (but a lesser amount on product. In addition, it has also been observed that the addition of water scavenging reagents can improve the yield. The alkylation/cyclization reaction is not affected by the particular choice of chiral entity or amino protecting group present in III-A.

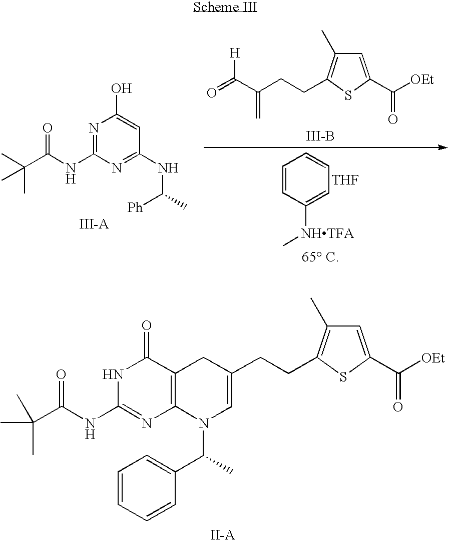

The two intermediates used in the alkylation/cyclization reaction of Scheme III, III-A and III-B, were prepared according to Schemes IV and V and VI and VII respectively. The “enal” intermediate II-B having structure: 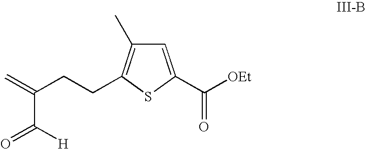
was obtained by treating aldehyde intermediate IV-A having structure: 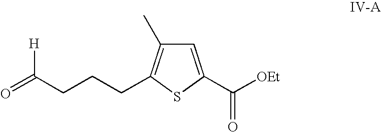
with Eschenmoser's reagent according to Scheme IV and Example IV. The reaction is performed in the presence of a base to neutralize acid formed.

Scheme V depicts a palladium-catalyzed coupling of commercially available bromothiephene V-A (Albemarle) having structure: 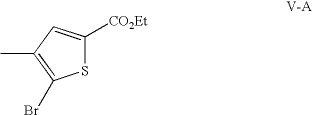
with 3-buten-1-ol (homoallylic alcohol). The initial product of the coupling is a vinyl-thiophene intermediate having a pendant alcohol moiety. Coupling of an alkene such as buten-1-ol to the thiophene can occur in two regiochemically distinct ways giving rise to major and minor regioisomers:

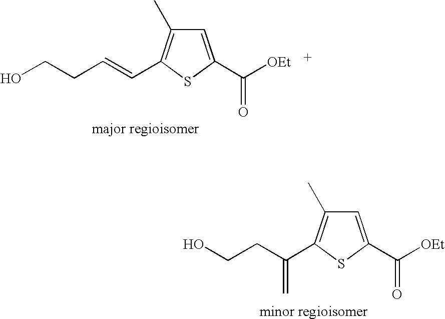

Subsequent double bond isomerization yields the major and minor diastereomers shown in Scheme V. The combined yield of the two aldehydes is 85% with 90% regioselectivity for the desired regioisomer, IV-A.

Scheme VI depicts the protection of the pyrimidine amino group of intermediate VI-A having structure:

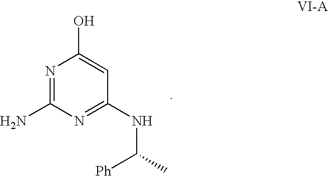

A preferred protecting agent is trimethylacetic anhydride, (abbreviated Piv₂O). The primary amino group of VI-A reacts with one electrophilic carbonyl moiety of Piv₂O, leading to formation of chiral N-Piv aminopyrimidinone III-A having structure:

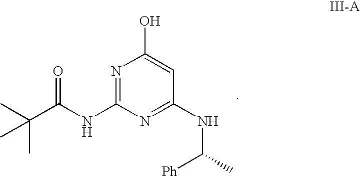

One equivalent of acid is co-produced during protection and is effectively neutralized by the base NEt₃. The latter base is also effective because it does not compete with amine moiety on the pyrimidine ring.

Scheme VII depicts the addition of a chiral amine to commercially available chloro pyrimidine VII-A having structure:

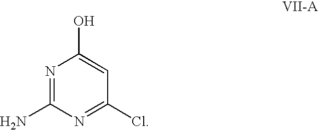

The amino moiety of chiral (S)-(−)-α-methylbenzylamine displaces the leaving group chloride from VII-A to make a intermediate VI-A having structure:

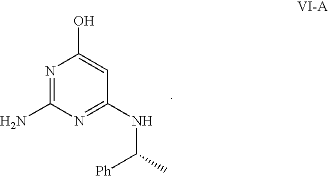

A number of commercially available chiral amines (or HCL salt precursors) may be used in place of (S)-(−)-α-methylbenzylamine, including (R)-(+)-α-methylbenzylamine which has been shown to influence the stereoselectivity of addition of H₂ in Scheme II.

Schemes VIII-X depict the final steps in the complete synthesis of one embodiment of the present invention. These steps comprise the deprotection of I-B and its subsequent functionalization.

Other aspects, advantages, and preferred features of the invention will become apparent from the Examples below.

### EXAMPLES

### Example I

Preparation of Intermediate I-B

TABLE I

I-B

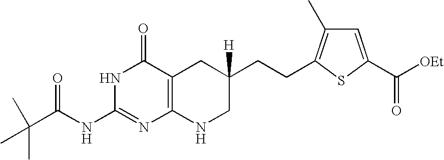

| | | | MW | | Wt. | density | Vol |
|---|---|---|---|---|---|---|---|
| Material | Source | Eq | (g/mol) | mmol | (g) | (g/mL) | (mL) |
| I-A | See Example | 1.00 | 550.3 | 0.363 | | | |
| | II | | | | | | |
| phenol | Aldrich | 3.0 | 94.0 | 1.09 | | | |
| TFA | | | | | | | 2 mL |
| (CH₂)₂Cl₂ | | | | | | | 6 mL |

Intermediate I-B was prepared according to Scheme I using the materials listed in Table I. A 50 mL round bottom flask equipped with a magnetic stirrer and condenser was charged with I-A (0.201 g, 0.363 mmol) in 6 mL of dichloroethane, phenol (0.10 g, 1.1 mmol), and 2 mL of TFA. The homogenous reaction was heated to reflux in an oil bath for 21 h. After this time, the reaction was cooled to room temperature and then quenched with 10 mL of distilled H₂O. The aqueous layer was extracted with 2×10 mL CH₂Cl₂. The combined organic layers were dried over MgSO₄, filtered, and the solvent removed in vacuo. The crude product was purified by flash chromatography in 1:1 EtOAc and hexanes to afford I-B as a white precipitate upon standing (0.145 g, 90%). ¹H NMR (300 MHz, DMSO-d₆) δ 11.18 (s, 1H), 10.49 (s, 1H), 7.47 (s, 1H), 6.40 (br s, 1H), 4.18 (q, J=7.20 Hz, 2H), 3.24 (m, 1H), 2.78 (m, 3H), 2.08 (s, 3H), 1.91 (m, 1H) 1.54 (m, 3H), 1.19 (t, J=7.20 Hz, 3H), 1.11 (s, 9 H); MS (ES, argon) m/z 447.00, [(M+H⁺) calcd for C₂₂H₃₀N₄O₄S: 446.56].

Proof of Stereochemistry

In a previous manufacturing route (“previous route”) for compound 1, the desired (S)-configuration was produced at carbon 6 via alkylation of an N-acyl (S)-oxazolidinone intermediate X—(S). This stereochemical outcome was expected based on well-known Evans oxazolidinone chemistry (Heathcock et al.; J. Org Chem. 1990, 55, 173; Heathcock et al.; J. Org Chem. 1990, 56, 5747.; Hayashi, K. et al.; Tetrahedron Left. 1991, 32, 7287; Evans et al.; J. Am. Chem. Soc. 1982, 104, 1737; Evans et al.; J. Am. Chem. Soc. 1981, 103, 2127). This same approach was described in the following scientific publication to produce the GARFT inhibitor LY309887. Tetrahedron Letters, 1997, 38, 735 (in the aforementioned paper, (6R)-stereochemistry is desired and so the corresponding (R)-oxazolidinone intermediate below is utilized): 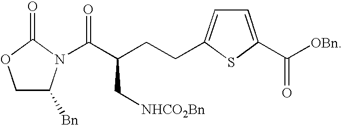
A) Synthesis of I-B-(S) from Intermediate X-(S) (Known Stereochemistry):

The previous manufacturing route used the intermediate X-(S) to prepare the subsequent intermediates VIII-A and I-B-(S), which are also intermediates in the present invention. Previous route: 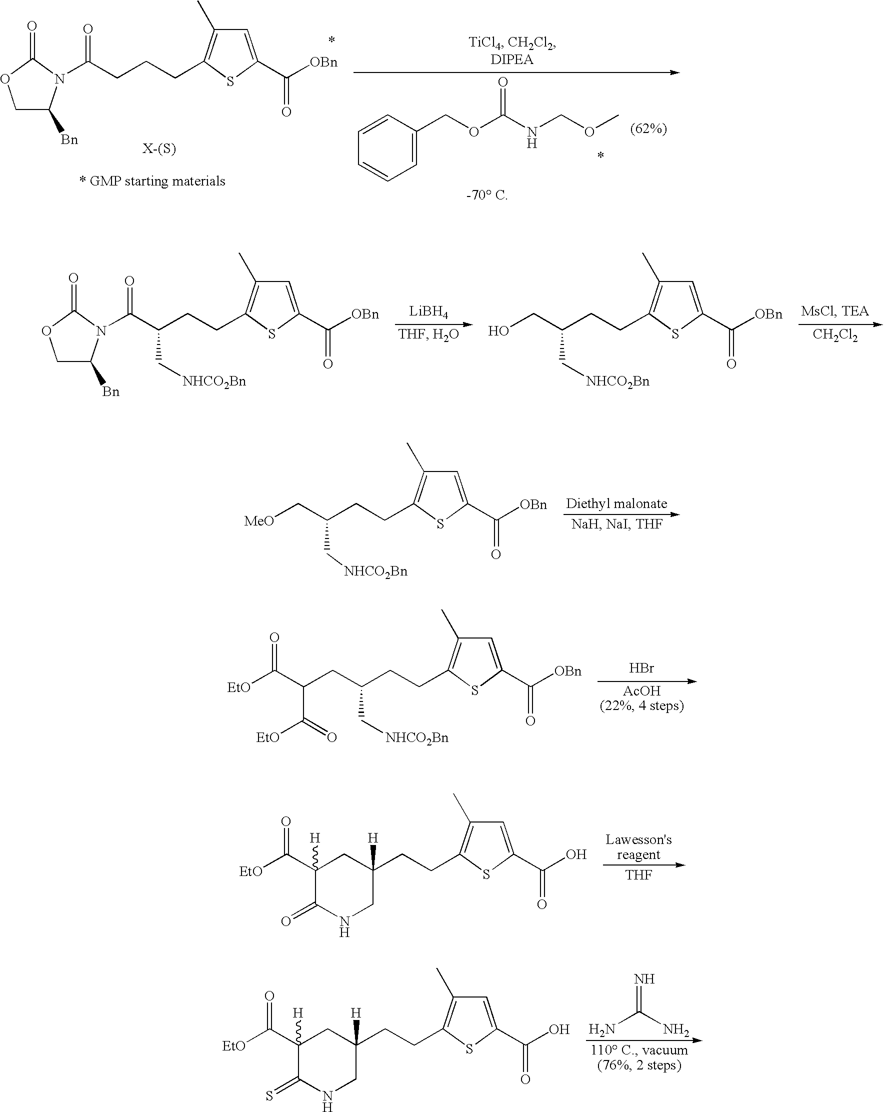 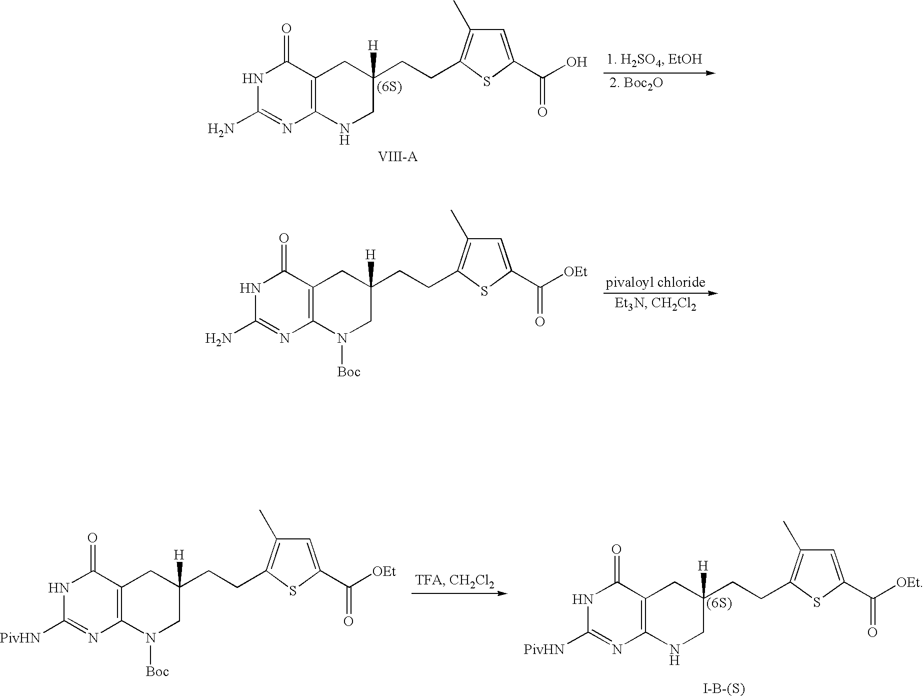
B) Synthesis of a 1:1 (Racemic) of I-B-(S) and I-B-(R) Sample and Chiral Analysis:

A sample of 1:1 (racemic) of I-B-(S) and I-B-(R) was synthesized using a further alternate route (depicted below as “alternate route”). A chiral HPLC method was developed to effectively separate the peaks corresponding to enantiomers I-B-(S) and I-B-(R). The HPLC method developed utilized a Chiralcel OJ-R column and 67/33 H₂O-ACN mobile phase (40° C., 40 minute method, UV detection at 235 nm), and the following two peaks were resolved: t=25.7 minutes and t=32.7 minutes. By comparison with the known I-B-(S) sample described in step A) above, the t=25.7 minute peak was determined to correspond to I-B-(S).

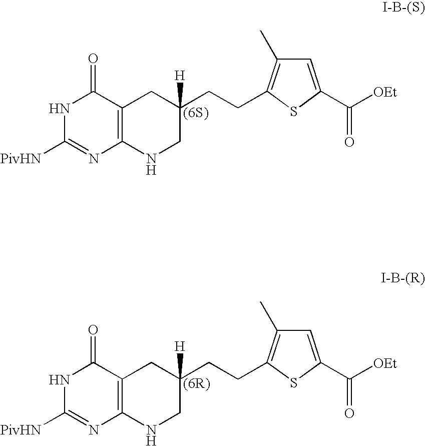

Alternate Route: 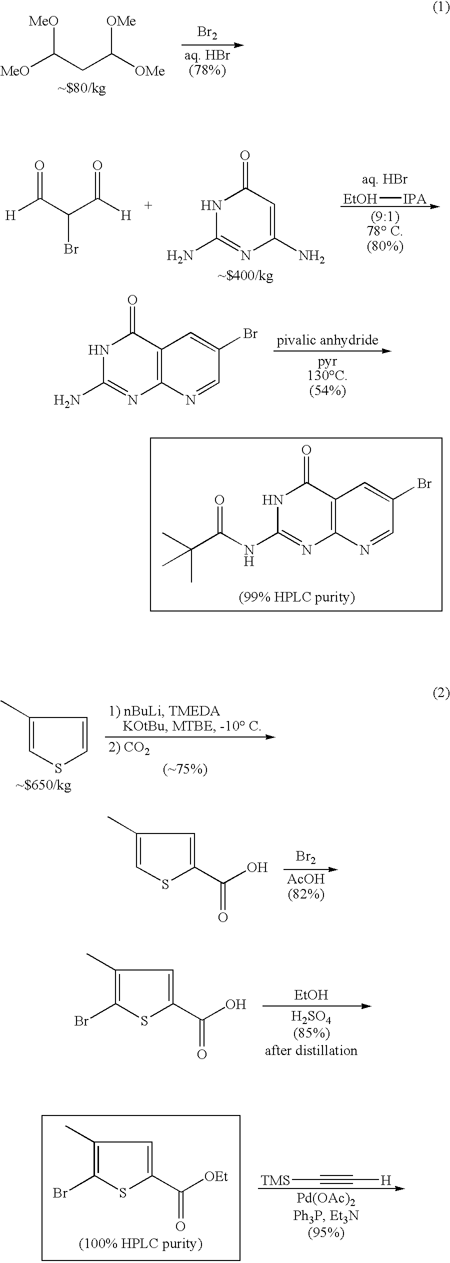 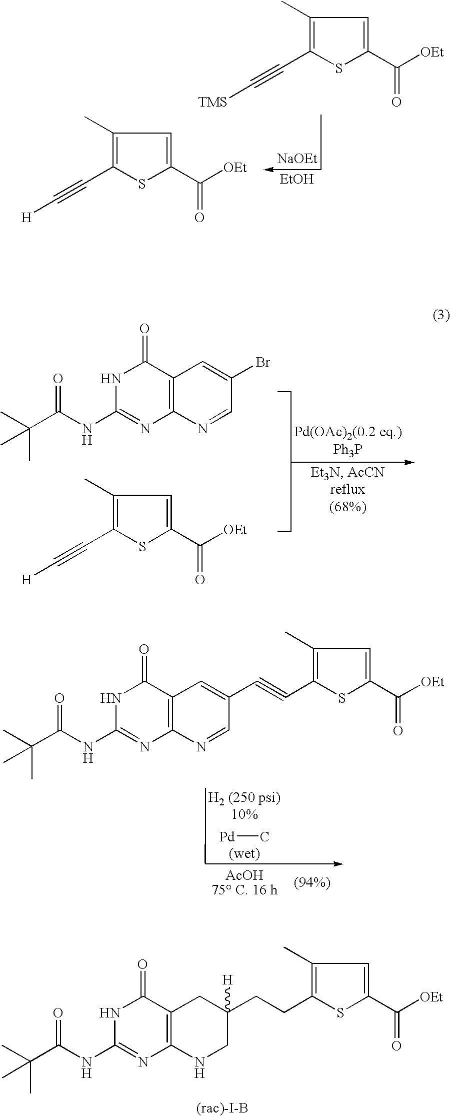
C) Determination of Predominant (6S) Stereochemistry for New Route Three Intermediates via Conversion to I-B-(S)/I-B-(R):

A representative sample of I-A-(S) and I-A-(R) produced via the present synthetic route was converted to the corresponding mixture of I-B-(S)/I-B-(R) in the manner depicted below

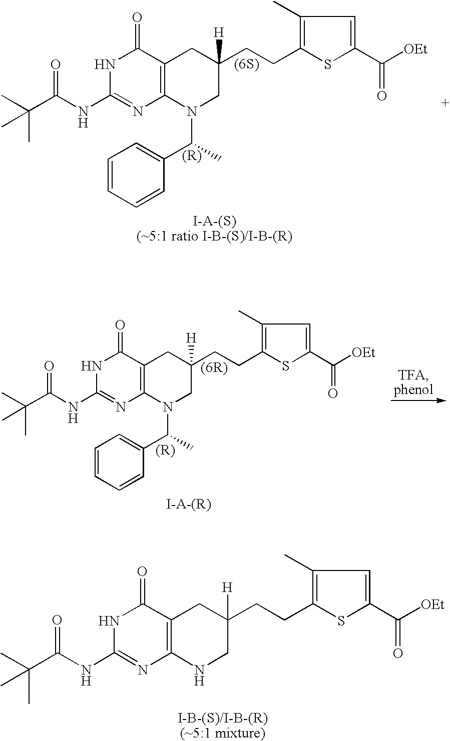

The resulting mixture of I-B-(S)/I-B-(R) was analyzed using the chiral HPLC method referenced in step B) above. The results of this analysis showed that peaks at t=25.8 minutes and t=32.9 minutes were in a approximately 5:1 ratio respectively via integration. Since the t=25.8 peak is known to correspond to (6S) configuration (steps A) and B) above), it was therefore concluded that the present conversion from II-A to I-A-(S)/I-A-(R) gives the desired (6S) configuration at carbon 6 as the predominant isomer.

### Example II

Preparation of Intermediate I-A

TABLE II

I-A

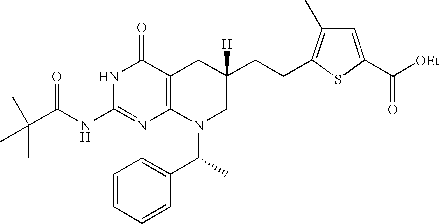

| | | | MW | | Wt. | density | Vol |
|---|---|---|---|---|---|---|---|
| Material | Source | Eq. | (g/mol) | mmol | (g) | (g/mL) | (mL) |
| II-A | See Scheme III | 1.00 | 548 | 14.1 | 7.7 | | |
| 5% | Johnson | 0 40 | | | 3.1 | | |
| Pd/C | Matthey | | | | | | |
| | Type: | | | | | | |
| | A405032-5 | | | | | | |
| EtOH | | | | | | | 140 |

Intermediate I-A was prepared according to Scheme II using the materials and amounts listed in Table II.

Engineering Technical Equipment (300 mL stirred H₂ Parr bomb reactor) was used for the hydrogenation of II-A. Intermediate II-A (7.7 g, 14.1 mmol) was dissolved in 100 mL of absolute ethanol and then placed in the reactor. 5% Pd/C (3.1 g) was slurried with 40 mL of absolute ethanol and added to the reactor. The reactor was sealed and purged 3 times with argon and then charged 2 times with hydrogen. The pressure was adjusted to 100 psi of H₂ and the reactor heated to 60° C. The reaction was stirred with a mechanical stirrer and the stir rate was set to 1000 RPM. After 2.5 h, an HPLC sample was taken and analysis on Prodigy 3 □m 4.6×100 mm (0.1% TFA/H2O; 0.1% TFA/ACN; isocratic) indicated >90 conversion to desired product. Reaction was allowed to continue overnight. After this time the heterogeneous reaction was filtered through celite 3 times and washed with EtOAc. The filtrate was concentrated in vacuo to afford a light tan, glassy solid that was put on high vacuum over night to afford I-A (6.03 g, 78%). ¹H NMR (300 MHz, CDCl₃) δ 8.25 (br s, 1H), 7.45 (s, 1H), 7.33 (m, 5H), 6.22 (q, J=6.9 Hz, 1H), 4.34 (q, J=6.9 Hz, 2H), 3.14-3.09 (br dd, J=12.6, 1.8Hz, 1H), 4.15-4.10 (m, 1H), 2.36 (dd, J=4.4, 7.5 Hz, 1H), 2.19 (dd, J=5.0 6.9 Hz, 1H) 2.15 (s, 3H), 1.12 (d, J=6.6 Hz, 3H); MS (ES, argon) m/z 551.10, [(M+H⁺) calcd for C₃₀H₃₈N₄O₄S: 550.71].

### Example III

Preparation of Intermediate II-A

TABLE III

II-A

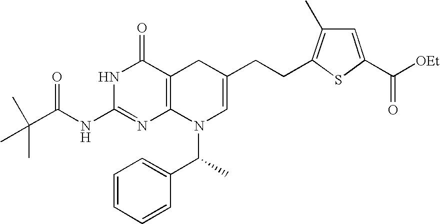

| | | | MW | | Wt. | density | Vol |
|---|---|---|---|---|---|---|---|
| Material | Source | Eq. | (g/mol) | mmol | (g) | (g/mL) | (mL) |
| III-A | See Exam- | 1.00 | 314 | 4.77 | 1.5 | | |
| | ple VI | | | | | | |
| III-B | See Exam- | 0.80 | 253 | 3.81 | 0.96 | 1.0 M | 530 |
| | ple IV | | | | | | |
| N | Aldrich | 0.20 | 107 | 0.95 | 0.10 | | |
| methyl- | | | | | | | |
| aniline | | | | | | | |
| TFA | Aldrich | 0.20 | 114 | 0.95 | 0.11 | 1.480 | 0.073 |
| THF | Aldrich | | | | | | 9.96 |

Intermediate II-A was prepared according to Scheme III using the materials and amounts listed in Table III.

A 3-neck 50 mL round bottom flask equipped with a condenser, an internal temperature probe (J-Kem), was charged under an inert atmosphere of argon with III-A (1.5 g; 4.77 mmol) and III-B (0.96 g, 3.81 mmol) as a solution in 9.0 mL of anhydrous THF. To this homogenous solution was added a 0.96 mL of 1 M solution of N-methylaniline.TFA salt in anhydrous THF via syringe. After addition was complete, the reaction was submerged in an oil bath. After 55 minutes the temperature of the reaction reached 65° C. Reaction was monitored by HPLC. After two hours of heating at 65° C., the reaction was complete. The orange homogenous reaction is cooled to room temperature over 0.5 h and then the volatiles stripped off in vacuo. The crude product was loaded onto a column and purified with 1:1 EtOAc-hexanes mobile phase to afford a yellow residue that when put on house vacuum. The residue turned into a glassy solid on high vacuum to afford II-A (1.56 g, 75%). ¹H NMR (300 MHz, CDCl₃) δ 7.79 (br s, 1H), 7.24 (s, 1H), 7.42-7.26 (br m, 5H), 5.73 (q, J=7.20 Hz, 1 H), 5.25 (br s, 1H), 4.14 (q, J=7.20 Hz, 2H), 3.13 (br s, 2H), 2.62 (t, J=7.50 Hz, 2 H), 2.00 (t, J=7.20 Hz, 2H), 1.89 (s, 3H), 1.74 (br m, 1H), 1.31 (t, J=7.20 Hz, 3 H), 1.12 (s, 9H); MS (ES, argon) m/z 549.10, [(M+H⁺) calcd for C₃₀H₃₆N₄O₄S: 548.70].

### Example IV

Preparation of Intermediate III-B

TABLE IV

| | | | | | | | III-B |
|---|---|---|---|---|---|---|---|
| 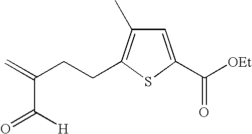 | | | | | | | |
| | | | MW | | Wt. | density | Vol |
| Material | Source | Eq. | (g/mol) | mmol | (g) | (g/mL) | (mL) |
| IV-A | See Example V | 1.00 | 24 | 4.84 | 1.16 | | 40 |
| N,N- | Aldrich, 98% | 2.3 | 185.01 | 11.0 | 2.0 | | |
| dimethylmethyleneammonium | | | | | | | |
| iodide (Eschenmoser's Salt) | | | | | | | |
| Et₃N | | 15 | 101.19 | 72.1 | | 0.726 | 10.0 |

Intermediate III-B was prepared according to Scheme IV using the materials listed in Table IV.

A flask containing IV-A (1.16 g, 4.84 mmol) was charged with Et₃N (10.0 mL, 72.1 mmol) and N,N-dimethylmethyleneammonium iodide (Eschenmosher's salt, C₃H₈NI) (2.0 g, 11.0 mmol). The heterogeneous reaction mixture became homogenous upon stirring for approx. 10-15 min. The homogenous solution was stirred for 2 h at which time HPLC (TFASH) analysis indicated full consumption of starting material. The reaction was diluted with 50 mL of CH₂Cl₂ and poured into a separatory funnel. The aqueous layer was extracted with 2×50 mL of CH₂Cl₂. The combined organic layers were washed with 40 mL of aqueous 5% NaHCO₃. The organic layer was dried over MgSO₄, filtered, and the filtrate was poured into a pad of silica gel and washed with 100 mL of EtOAc. The solvent was removed in vacuo and the crude product was purified by flash chromatography in 10% EtOAc in hexanes to afford (0.84 g, 69%) of product III-B as a yellow oil. ¹H NMR (300 MHz, CDCl₃) δ 7.40-7.23 (m, 5H), 5.83 (br d, J=7.2 Hz, 1H), 5.09 (s, 2H), 4.67 (m, 1H), 3.05 (s, 3H), 2.95 (s, 3H), 1.32 (d, J=6.8 Hz, 3H); ¹³C NMR (300 MHz, CDCl₃) δ 194.5, 162.7, 148.6, 145.7, 136.4, 135.7, 134.3, 129.6 61.7, 29.9, 27.0, 14.7, 13.9; IR: 2929, 1698, 1444, 1372, 1281, 1180, 1070, 864, 752 cm⁻¹; MS (ES, argon) m/z 253.10, [(M+H⁺) calcd for C₁₃H₁₆O₃S: 252.33].

### Example V

Preparation of Intermediate IV-A

TABLE V

| | | | | | | | IV-A |
|---|---|---|---|---|---|---|---|
| 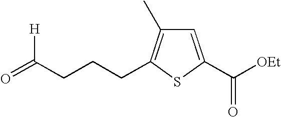 | | | | | | | |
| | | | MW | | | density | vol |
| Material | Source | Eq. | (g/mol) | mmol | Wt. | (g/mL) | (mL) |
| Ethyl 5- | Albemarle | 1.00 | 249.1 | 40.2 | 10.0 | | |
| bromo, 4- | Fine | | | | | | |
| methyl, 2- | Chemicals | | | | | | |
| thiophene | | | | | | | |
| carboxylate | | | | | | | |
| V-A | | | | | | | |
| 3-buten-1-ol | Aldrich, | 1.2 | 72.1 | 48.2 | 4.2 | 0.838 | 4.1 |
| | 96% | | | | | | |
| Pd(OAc)₂ | Johnson | 0.025 | 224 | 1.0 | 0.22 | | |
| | Matthey | | | | | | |
| NaOAc | Aldrich, | 1.25 | 82.0 | 50.0 | 4.1 | | |
| | 99% | | | | | | |
| LiCl | Aldrich, | 3.00 | 42.39 | 120 | 5.1 | | |
| | 99% | | | | | | |
| t-Bu₄NCl | Aldrich, | 0.50 | 277.9 | 20.1 | 4.6 | | |
| | 99% | | | | | | |
| DMF | Aldrich, | | | | | | 80 |
| | 99% | | | | | | |

Intermediate IV-A was prepared according to Scheme V using the materials and amounts listed in Table V.

A round-bottom flask containing 80 mL of anhydrous DMF under an inert atmosphere of argon was charged with bromothiophene, V-A (10.0 g; 40.2 mmol), NaOAc (4.1 g, 50.0 mmol), LiCl (5.1 g, 120 mmol), t-Bu₄NCl (4.6 g, 20.1 mmol), and 3-buten-1-ol (4.2 g, 48.2 mmol). The heterogeneous mixture was sparged with argon via a gas dispersion tube for 0.5 hour. After this time, Pd(OAc)₂ (0.22 g, 1.0 mmol) was added to the mixture and the reaction was heated to 70° C. for 6.5 hours. The reaction was then cooled to room temperature and 500 mL of EtOAc was added to wash the contents of the round-bottom flask into a 1 L separatory funnel. The organic layer was washed with 3×500 mL of deionized water. The organic phase was separated and dried over MgSO₄ and then filtered via a fritted funnel. The organic phase was then concentrated in vacuo to afford IV-A along with the regioisomer shown in Scheme V as a dark brown oil. The crude product was purified by flash chromatography in 12% EtOAc in hexanes to afford a reddish oil. The purified product IV-A (8.2 g, 85%) was carried on to the next step. ¹H NMR (300 MHz; CDCl₃) δ 9.78 (s, 1H), 7.50 (s, 1H), 4.30 (q, J=9 Hz, 2H), 2.79 (t, J=6 Hz, 2H), 2.53 (q, J=6 Hz, 2H), 2.15 (s, 3H), 1.98 (m, 2H), 1.35 (t, J=9 Hz, 3H); ¹³C NMR (300 MHz, CDCl₃) δ 201.8, 162.7, 146.0, 136.6, 134.8, 133.9, 61.2, 43.2, 27.8, 23.6, 14.7, 13.9; IR: 2934, 1699, 1444, 1389, 1373, 1281, 1177, 1095, 1065, 119, 865, 752 cm⁻¹.

### Example VI

Preparation of Intermediate III-A

TABLE VI

| | | | | | | | III-A |
|---|---|---|---|---|---|---|---|
| 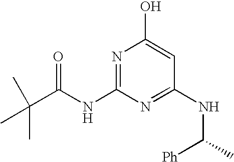 | | | | | | | |
| | | | MW | | Wt. | density | Vol |
| Material | Source | Eq. | (g/mol) | mmol | (g) | (g/mL) | (mL) |
| VI-A | See Example | 1.00 | 230.27 | 130.0 | 30.0 | | |
| | VII | | | | | | |
| Trimethyl- | Aldrich | 3.00 | 186.25 | 390.0 | 72.5 | 0.918 | 79 |
| acetic acid | | | | | | | |
| anhydride | | | | | | | |
| Et₃N | | 4.00 | 101.19 | 512 | 52.3 | 0.726 | 72 |
| DMF | | | | | | | 120 |

Chiral N-Piv Aminopyrimidone III-A was prepared according to the method of Scheme VI using the materials and amounts listed in Table VI.

Compound VI-A (30.0 g; 130 mmol) was charged into a 500 mL round-bottom flask equipped with a stir bar and condenser submerged in an oil bath under an inert atmosphere of nitrogen. To this flask was added Et₃N (72 mL, 512 mmol), DMF (120 mL), and trimethylacetic acid anhydride (79 mL, 390 mmol). The reaction was a thick slurry. The oil bath was heated to 85° C. and the reaction became less viscous. Reaction was heated for 4.5 hours. After this time the reaction was cooled to room temperature. The reaction was a slurry and was filtered through a course glass frit funnel and the filter cake was pulled dry under vacuum. The cake was taken up in 400 mL of MTBE and 200 mL of H₂O. The product was extracted with 3×400 mL of MTBE. Each wash was collected separately and concentrated in vacuo to afford three fractions of solid having 90.34 g, 4.6 g, 1.0 g of crude product as an off-white solid. Each of the fractions were charged separately into a round bottom flask equipped with a stir bar. To the flasks were added 100 mL of EtOAc and heated to 85° C., cooled to room temperature and then 400 mL of hexanes were added and the contents heated to reflux to give a slurry. This slurry was cooled to room temperature and stirred over night. After this time, the slurry was filtered through Whatman #2 and washed with 3×50 mL of hexanes and the filter cake was pulled dry under house vacuum to afford the desired compound III-A as a white solid (36.7 g, 90%). ¹H NMR (300 MHz, DMSO-d₆) δ 11.25 (s, 1H), 10.42 (s, 1H), 7.30 (m, 5H), 7.22 (m, 1H), 4.73 (br s, 1H), 1.41 (d, J=6.90 Hz, 3H), 1.21 (s, 9H); ¹³C NMR (300 MHz, DMSO-d₆) δ 180.8, 161.7, 160.4, 150.3, 144.3, 128.0, 126.3, 125.5, 80.5, 50.2, 25.9, 23.0; MS (ES, argon) m/z 315.00, [(M+H⁺) calcd for C₁₇H₂₂N₄O₂: 314.38].

### Example VII

Preparation of Intermediate VI-A

TABLE VII

| | |
|---|---|
| | VI-A |
| 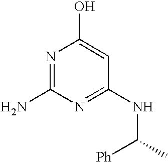 | |

| | | | MW | | Wt. | density | Vol |
|---|---|---|---|---|---|---|---|
| Material | Source | Eq. | (g/mol) | mmol | (g) | (g/mL) | (mL) |
| 2-amino-6-chloro-4- | Aldrich | 1.00 | 145.6 | 45.0 | 6.54 | | |
| pyrimidinol, VII-A | (sold as hydrate) | | | | | | |
| (S)-(−)-α- | Aldrich >99% | 2.6 | 121.2 | 119 | 14.5 | 0.940 | 15.4 mL |
| methylbenzylamine | | | | | | | |
| 1-ethyloxyethanol | | 15 | 101.19 | 72.1 | | 0.726 | 30 mL |

Intermediate VI-A was prepared according to the method of Scheme VII using the materials and amounts listed in Table VII. The corresponding enantiomer is available by substituting commercially available (R)-(+)-α-methylbenzylamine for (S)-(−)-α-methylbenzylamine. A variety of chiral amines may also be employed and representative examples are listed elsewhere.

2-Amino-6-chloro-4-pyrimidol, VII-A, (6.54 g; 45.0 mmol) was charged into a 100 mL round-bottom flask equipped with a stir bar and condenser submerged in an oil bath under an inert atmosphere of nitrogen. To this flask was added (S)-(−)-α-methylbenzylamine (15.4 mL, 121.2 mmol), and 2-ethoxyethanol (30 mL). The oil bath was heated to 140° C. and the reaction was heated overnight. After this time the hot reaction was poured into 150 mL of ice water. The reaction formed a white cloudy heterogeneous mixture and this was allowed to sit for 30 mins. Attempts to filter the mixture proved unsuccessful. The aqueous layer was extracted with 3×100 mL of EtOAc. The combined organic layers were dried over MgSO₄ filtered, and the solvent was removed in vacuo to afford a brown residue that was diluted with 250 mL of EtOAc and washed with 4×100 mL of 10% aqueous citric acid. The organic and aqueous layers were partitioned and the aqueous layer was brought to pH 5 with saturated NaHCO₃. The aqueous layer was extracted 3×150 mL of EtOAc, dried over MgSO₄, filtered, and the solvent was removed in vacuo to afford compound VI-A as a yellow solid (5.6 g, 54%). ¹H NMR (300 MHz, DMSO-d₆) δ 3.78 (q, J=6.78, 1H), 2.98 (s, 3H), 2.82 (s, 3H), 1.07 (d, J=6.78, 3H); MS (ES, argon) m/z 231.10, [(M+H⁺) calcd for C₁₂H₁₄N₄O: 230.27].

1. A method of producing a compound or salt of formula (I)-S: 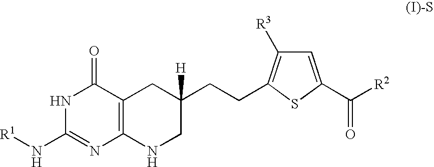
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
said method comprising:
(i) treating a compound or salt of formula (II)-S: 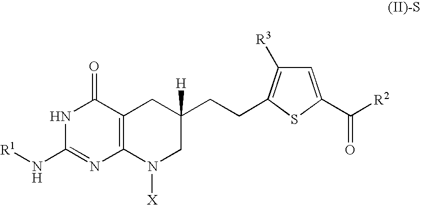
with an acid in an inert solvent, wherein X is —CHR⁵R⁶ and comprises a chiral entity; R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁶ is an aryl group.

2. A method of producing a compound or salt of formula (I)-R: 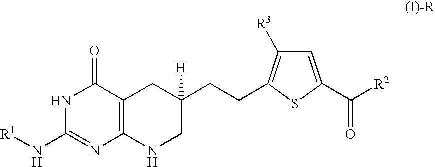
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups,;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
said method comprising:
(i) treating a compound or salt of formula (II)-R: 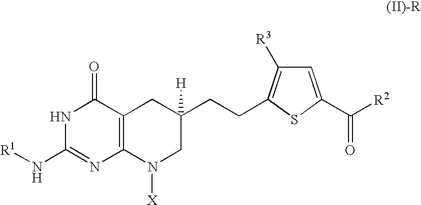
with an acid in an inert solvent,
wherein X is —CHR⁵R⁶ and comprises a chiral entity;
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

3. A compound or salt having formula (III): 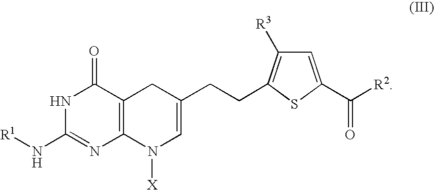
wherein
R¹ is H or an amino protecting group;
R² is —OR⁴ or an amino acid moiety;
R³ and R⁴ are each, independently, H or a moiety selected from the group consisting (C₁-C₆)alkyl, (C₃-C₆)cycloalkyl, (C₅-C₆)cycloalkenyl, aryl, heterocycle, and (C₂-C₆)alkenyl, said moiety being optionally substituted with 1 to 3 independently selected Y¹ groups,;
each Y¹ is independently selected from the group consisting of halogen, cyano, nitro, azido, —OH, —NH₂, -Z¹-(C₁-C₆)alkoxy, -Z¹-(C₁-C₆)alkylamino, -Z¹-(C₁-C₆)dialkylamino, -Z¹-(C₁-C₆)alkyl, -Z¹-(C₂-C₆)alkenyl, -Z¹(C₂-C₆)alkynyl, -Z¹-(C₁-C₆)haloalkyl, -Z¹-(C₁-C₆)haloalkoxy, -Z¹-(C₃₋C₆)cycloalkyl, -Z¹-aryl, and -Z¹-heterocycle, wherein Z¹ is a bond, —C(O)—, —OC(O)—, or —NHC(O)—;
X is —CHR⁵R⁶ and comprises a chiral entity;
R⁵ and R⁶ are each, independently selected from the group consisting (C₁-C₆)alkyl, aryl, —(CH₂)ₘOR⁸ and (C₁-C₆)heteroalkyl, said R⁵ and R⁶ being optionally substituted with 1 to 3 independently selected Y² groups; wherein Y² is independently selected from the group consisting of halogen, cyano, nitro, azido, —OH, —NH₂, -Z²-(C₁-C₆)alkoxy, -Z²-(C₁-C₆)alkylamino, -Z²-(C₁-C₆)dialkylamino, -Z²-(C₁-C₆)alkyl, -Z²-(C₂-C₆)alkenyl, -Z²-(C₂-C₆)alkynyl, -Z²-(C₁-C₆)haloalkyl, -Z²-(C₁-C₆)haloalkoxy, -Z²-(C₃₋C₆)cycloalkyl, -Z²-aryl, and -Z²-heterocycle, wherein Z² is a bond, —C(O)—, —OC(O)—, or —NHC(O)—, m is an integer 0-2 and R⁸ is an aryl group.

4. A method of producing a compound or salt according to claim 3 having formula III: 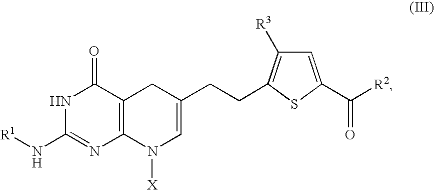
comprising:
(i) treating a compound or salt of formula (IV): 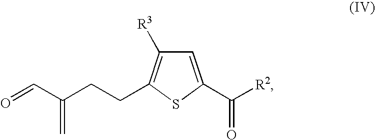
with a compound or salt of formula (V): 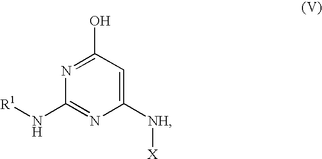
and with an acid in a solvent.

5. The method according to claim 4, wherein said acid is an acid salt of a secondary amine and the acid is present in a catalytic amount.

6. The method according to claim 8 wherein the acid is the trifluoroacetic acid salt of N-methyl aniline.

7. The method according to claim 2, wherein X is 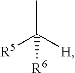
R⁵ is a (C₁-C₃)alkyl, optionally substituted with 1-3 independently selected Y² groups;
and R⁶ is an aryl, optionally substituted with 1-3 independently selected Y² groups.

8. The method according to claim 7, wherein R⁵ is CH₃ and R⁶ is phenyl.

9. The method according to claim 1, wherein R¹ is —C(O)R⁷ and R⁷ is selected from the group consisting of (C₁-C₆)alkyl, alkaryl and aryl.

10. The method according to claim 9 wherein R¹ is

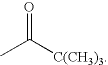

11. The method according to claim 1, wherein R² is OR⁴, wherein R⁴ is (C₁-C₆)alkyl, optionally substituted with 1 to 3 independently selected Y¹ groups.

12. The method according to claim 11, wherein R⁴ is —CH₂CH₃.

13. The method according to claim 1, wherein R² is [chemical structure]
